(19) United States
(12) Patent Application Publication
(10) Pub. No.: US 2013/0143822 A1
(43) Pub. Date: Jun. 6, 2013

(54) AGENTS FOR TREATING ALZHEIMER'S DISEASE

(71) Applicants: Susanne Aileen Funke, Titz (DE); Luitgard Nagel-Steger, Langenfeld (DE); Dirk Bartnik, Koeln (DE); Olexandr Brener, Duesseldorf (DE); Torsten Sehl, Juelich (DE); Katja Wiesehan, Hohenschaeftlarn (DE); Dieter Willbold, Juelich (DE)

(72) Inventors: Susanne Aileen Funke, Titz (DE); Luitgard Nagel-Steger, Langenfeld (DE); Dirk Bartnik, Koeln (DE); Olexandr Brener, Duesseldorf (DE); Torsten Sehl, Juelich (DE); Katja Wiesehan, Hohenschaeftlarn (DE); Dieter Willbold, Juelich (DE)

(21) Appl. No.: 13/695,682

(22) PCT Filed: Apr. 9, 2011
(86) PCT No.: PCT/DE2011/000389

§ 371 Date: Nov. 1, 2012

(30) Foreign Application Priority Data
May 5, 2010 (DE) 10 2010 019 336.4

(51) Int. Cl. C07K 14/00; C07K 7/08

(52) U.S. Cl. CPC C07K 14/00; C07K 7/08
USPC 514/17.8; 530/327; 530/324; 536/23.1; 435/320.1

(57) ABSTRACT

Agents for treating Alzheimer's disease comprising a peptide according to sequence no. 1 which binds to Aβ oligomers and thus results in the healing or alleviation of Alzheimer's disease. In further embodiments peptides are provided which contain a sequence no. 1, but have preceding sequence sections which allow the peptide to be secreted. For the purpose of gene therapy, corresponding DNA sequences and vectors, in particular according to sequences 3 to 6, are provided.

### BACKGROUND OF THE INVENTION

The invention relates to agents for treating Alzheimer's disease,

Alzheimer's disease (AD) is the most common form of dementia and today affects more than 60% of the estimated 24 million people suffering from dementia worldwide. A key pathological feature of AD is the formation of senile or amyloid plaques, composed of the Aβ peptide, and neurofibrillary tangles of the tau protein. The Aβ peptide is created by the activities of at least two different proteases from a precursor protein, the amyloid precursor protein (APP). This protein is localized in the cell wall of neurons. The proteolytic degradation of APP and subsequent modification results in Aβ fragments of varying lengths and types, The amyloid cascade hypothesis was developed in the 1990s and posits that the deposition of Aβ in form of plaques is a central trigger of the symptoms of the disease. Freely diffusable Aβ oligomers are more toxic than the Aβ fibrils deposited in the plaques. According to recent papers, the plaques can be considered to be a reservoir for oligomeric Aβ, which colocalizes, with the destruction of synapses and neurons.

The aggregation of intraneuronal Aβ (Aβi) is considered to be a significant factor in the early pathogenesis of AD. It has not been conclusively clarified whether Aβi, which to a large degree consists of Aβ1-42, is secreted Aβ or reinternalized Aβ. However, indications of the second option are on the rise.

So far, only the symptoms of AD can be treated No approved medications are known, which can stop or reverse the disease process. The majority of substances being explored for AD treatment focus on extracellular Aβ, but not specifically on soluble Aβ oligomers. This would, however, be desirable in order to be able to stop the disease process at an early stage.

European patent 1379 546 B1 points out that various D-enantiomeric peptides bind to the β-amyloid peptide and may therefore be suitable for treating Alzheimer's disease. In particular the peptide according to claim 3, alternative e) disclosed in the document, also referred to as D3 peptide, modulates Aβ aggregation. The D3 peptide interacts with soluble Aβ oligomers. Surface plasmon resonance studies indicate that D3 preferentially binds soluble Aβ oligomers. In the APP transgenic mouse model. D3 reduces the number of senile plaques in the brain and the associated inflammatory processes.

So far, only palliative treatments are available for Alzheimer's disease. The causes cannot be treated as of yet, but there are extensive research efforts working on wide variety of treatment options. These frequently focus on preventing Aβ aggregation, for example by way of substances that bind to Aβ and thus make (further) aggregation impossible.

Substances are required which i) reduce toxic, soluble Aβ oligomers in vivo and ii) are not only effective outside, but also inside neurons.

It is therefore the object of the invention to provide agents which allow better therapeutic treatment of Alzheimer's disease.

The object was surprisingly achieved according to the invention by providing agents and a method for treating Alzheimer's disease.

The sequences listed in the sequence listing will be defined hereafter.

Sequence no. 1: L3 peptide, which according to the invention binds to Aβ oligomers.

Sequence no. 2: Peptide according to the invention which is listed by way of example and which comprises sequence no. 1, but also contains a sequence section which causes secretion through a cell membrane.

Sequence no. 1 DNA sequence coding for peptide no. 1.

Sequence no. 4: DNA sequence coding for peptide no. 2.

Sequence no. 5: Sequence coding for a vector which contains sequence no. 3 and codes for a structural unit that fluoresces.

Sequence no. 6: Sequence coding for a vector which contains sequence no. 4 and codes for a structural unit that fluoresces.

The peptides according to the invention are preferably L-enantiomers. The DNA sequences and vectors coding therefor likewise preferably code for L-enantiomers.

According to the invention, the peptide according to sequence no. 1 binds to the Aβ peptide, and more particularly to Aβ oligomers. It is therefore a pharmaceutical for treating Alzheimer's disease. The pharmaceutical for treating Alzheimer's disease can thus be composed of the peptide according to sequence no. 1 or of a substance containing the peptide according to sequence no. 1. The peptide according to sequence no. 1 has the property of binding better binding to the Aβ peptide than peptide D3. It allows both intracellular and extracellular use for treating Alzheimer's disease.

The peptide according to sequence no. 1 can be produced synthetically, for example using Merrifield synthesis and expression of DNA coding for sequence no. 1.

The peptide according to sequence no. 1 can also be used to produce a pharmaceutical for treating Alzheimer's disease.

The peptide according to sequence no. 1 thus binds to Aβ oligomers both intracellularly and extracellularly. This allows Alzheimer's disease to b treated both by intracellular and by extracellular action.

In one refinement of the invention, a protein is provided which contains a sequence section according to sequence no. 1, but which comprises a sequence section that codes for the function that the peptide is secretable, which is to say that it can pass through a cell membrane. These proteins can be exported from the cell. The peptide according to sequence no. 1 has better binding properties to Aβ than peptide D3. It allows both intracellular and extracellular treatment of Alzheimer's disease.

The sequence sections causing secretion are known to the person Skilled in the art. By way of example, a peptide according to sequence no. 2 can be provided as a secretable peptide that has the mentioned properties. The sequence section causing secretion which is used is preferably one which is of human origin or is identical to a human sequence. This has the advantage that an undesirable immune response to the secretion section can be prevented or suppressed when treating the person. The secretable peptides, containing a sequence section according to sequence no. 1, can pass through cell membranes and thus have a site of action that is located across the cell membrane.

The secretable peptides can also be produced by Merrifield synthesis or by expression of the corresponding DNA. These secretable peptides are pharmaceuticals. They can also be used to produce a pharmaceutical for treating Alzheimer's disease. The secretable peptides can be used intracellularly or extracellularly.

The peptides according to the invention in accordance with sequence nos. 1 and 2, as well as further secretable peptides that contain sequence fragments according to sequence no. 1, bind to the monomeric, oligomeric or fibrillary or plaque-like Aβ peptide. The peptides according to the invention bind particularly well to soluble oligomeric Aβ peptides. A particularly large effect was observed with Aβ peptides having the structural length Aβ1-42.

In a further advantageous embodiment of the invention, a DNA is provided which codes for a peptide according to sequence no. 1.

The DNA can be expressed intracellularly, so that a peptide according to sequence no. 1 is created, which is suitable for treating Alzheimer's disease. This DNA is therefore suited for gene therapy. The DNA coding for a peptide according to sequence no. 1 is a pharmaceutical that can be used in particular for treating Alzheimer's disease. It can also be used to produce a pharmaceutical for treating Alzheimer's disease.

A DNA according to sequence no. 3 is provided by way of example.

In a further preferred embodiment, a DNA is provided which codes for a peptide containing sequence no. 1, which comprises a sequence section that functionally codes for a secretability of the peptide. This DNA as well can be expressed intracellularly, so that a peptide according to sequence no. 2 is created, which is secretable and contains a section according to sequence no. 1, which is suitable for treating Alzheimer's disease. This DNA is therefore suited for gene therapy. The section of the DNA which is responsible for the secretion preferably codes for a human secretion sequence, The DNA coding for such a peptide is a pharmaceutical that can be used in particular for treating Alzheimer's disease. It can also be used to produce a pharmaceutical for treating Alzheimer's disease.

A DNA according to sequence no. 4 is provided by way of example.

In a further embodiment of the invention, vectors are provided which contain a DNA section that codes for a protein according to sequence no. 1. The vectors can also contain a DNA section coding for a protein according to sequence no. 1 which comprises a DNA sequence that functionally causes a secretion of the expressed DNA section or protein. The vectors can be used to intracellularly express peptides according to sequence no. 1 and secretable derivatives thereof, such as peptides according to sequence no. 2. The vectors can contain sections that functionally code for fluorescent structural components. By way of example, vectors according to sequence 5 or 6 can be provided.

The vectors according to the invention can be produced by methods known to persons skilled in the art starting from vectors available for purchase. These are pharmaceuticals, especially for treating Alzheimer's disease, and can be used to produce a pharmaceutical for treating Alzheimer's disease. Viral vectors are particularly well suited, because they can be used particularly well for human gene therapy, but also for other living beings, such as animals.

For gene therapy, the deoxyribonucleic acids coding for a peptide according to sequence no. 1, the deoxyribonucleic acids coding for a peptide according to sequence no. 1 comprising a sequence section for secretability, for example, for a peptide according to sequence no. 2, and vectors comprising the corresponding nucleic acids can also be used. By way of example, a DNA and a vector according to sequences 3 to 6 can be used. These are introduced into the body.

### EXAMPLE

L3 is expressed in cells of the central nervous system, for example in neurons or in cells, and is subsequently secreted and thus specifically leads to a reduction of the particularly toxic Aβ oligomers. This can be achieved using special viral vectors. Experiments were conducted in cell cultures. The expression of L3 was carried out both intracellularly and extracellularly.

### EXPERIMENTAL RESULTS

The figures show experimental results:

### BRIEF DESCRIPTION OF THE DRAWINGS

FIG. 1: shows a comparison of the binding preferences of L3 and D3 for Aβ oligomers;

FIG. 2: shows comparison results of the density gradient centrifugation of Aβ-42 without peptide, with L3 and with D3;

FIG. 3: shows a comparison of the hydrodynamic radius of Aβ1-42 particles with and without L3 at different times;

FIG. 4: is a Thioflavin T test and turbidimetric test for analyzing the aggregation behavior; and

FIG. 5: shows the ThT fluorescence intensity.

### DESCRIPTION OF THE PREFERRED EMBODIMENTS

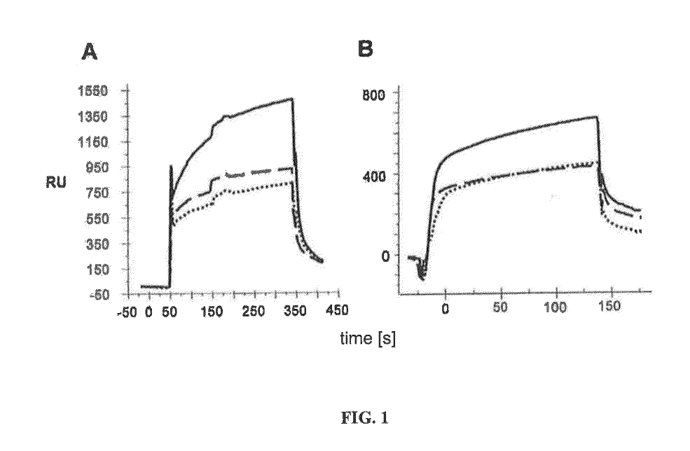

FIG. 1 shows the comparison of the preferential binding of L3 and D3 for Aβ1-42 oligomers. L3 is shown in section A and D3 is shown in section B of the figure. L3 exhibits stronger binding than D3. Aβ1-42 monomers (dashed lines), oligomers (solid lines) and fibrils (dotted line) were immobilized on a CM5 biosensor chip. Interaction analyses were carried out by means of surface pl s on resonance. RU: resonance units. In each case, 25 μl peptide solution (100 μg/ml) was injected. Both peptides exhibit very clear binding to Aβ1-42 oligomers, while L3 generally exhibits a higher maximum resonance than D3. The same results are also obtained for Aβ1-40 oligomers.

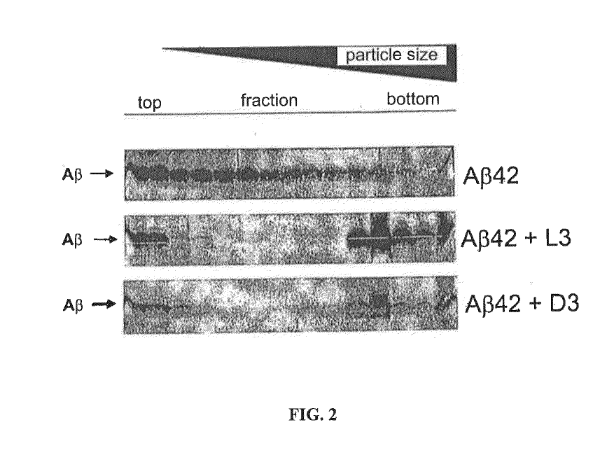

FIG. 2 shows the results of comparison of the density gradient centrifugation of Aβ1-42 without peptide, with L3 and with D3. L3 precipitates Aβ oligomers from complex mixtures of different Aβ forms. The size distributions of Aβ in solution and in Aβ-peptide mixtures were examined by way of sedimentation analysis on an iodixanol gradient (5-50%). The mixtures contained 125 μM Aβ and 125 mM peptide, respectively. After centrifugation, 14 fractions of 140 μl each were obtained from the surface by sequential pipetting and analyzed by means of denatured polyacrylamide gel electrophoresis SDS-PAGE and subsequent silver staining. The results show that peptides significantly reduce the content of Aβ oligomers, with L3 doing so to a greater degree than D3 at the analyzed time. Large aggregates form, which in subsequent experiments are described as amorphous, not fibrillary and not amyloidogenic.

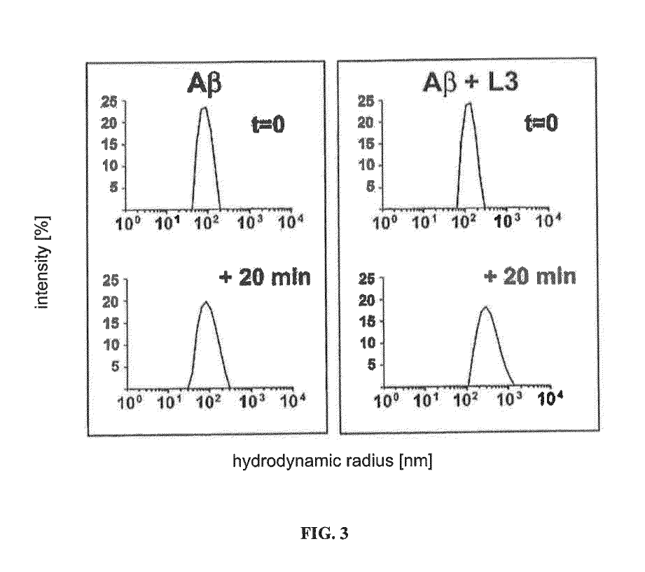

FIG. 3 shows the results of experiments on the comparison of the hydrodynamic radius of Aβ1-42 particles with and without L3 at different times. Dynamic light scattering is used to determine the hydrodynamic radius of particles in solution or suspension. A 5 μM sample of Aβ1-42 oligomeric particles was diluted by adding a 50 μM L3 sample on the one hand, and buffer (50 mM sodium phosphate, 100 mM NaCl, pH 7.4) on the other hand. The hydrodynamic radius of the Aβ1-42 particles with and without L3 was measured using a DynaPro light scattering system, immediately after the sample was prepared and after 20 minutes. A 655.6 nm laser (13 mW/58% laser intensity) was used. The measuring time was 2 seconds, and the measurement temperature was 25° C. Spherical sedimented particles were assumed for calculating the hydrodynamic radius. L3 is favorable in terms of the fast formation of large Aβ aggregates.

FIG. 4 shows the results of a Thioflavin T test and a turbidimetric test for analyzing the aggregation behavior of Aβ in the presence of L3. Both tests were prepared from joint stock solutions made of 25 μM Aβ (light bars) and 25 μM Aβ with 1 mM L3 (dark bars). In the ThT test, 5 μl of the solutions was mixed with 200 μl ThT solution (5 μM ThT; 50 mM glycerin pH 8.5) and measured at λₑₓ 440 nm and λₑₘ=490 nm in the fluorescence spectrometer. ThT is a dye which, when bound to regular fibrils, has higher fluorescence and therefore serves as a measure of the fibrillation. In the turbidimetric test, the clouding of the solution was measured in the UV/VIS spectrometer as a measure of the aggregation as absorption at 355 nm. L3 is favorable in terms of the fast development of large Aβ aggregates which have no fibrillary structure and are thus negative in the Thiofiavin T (ThT) test.

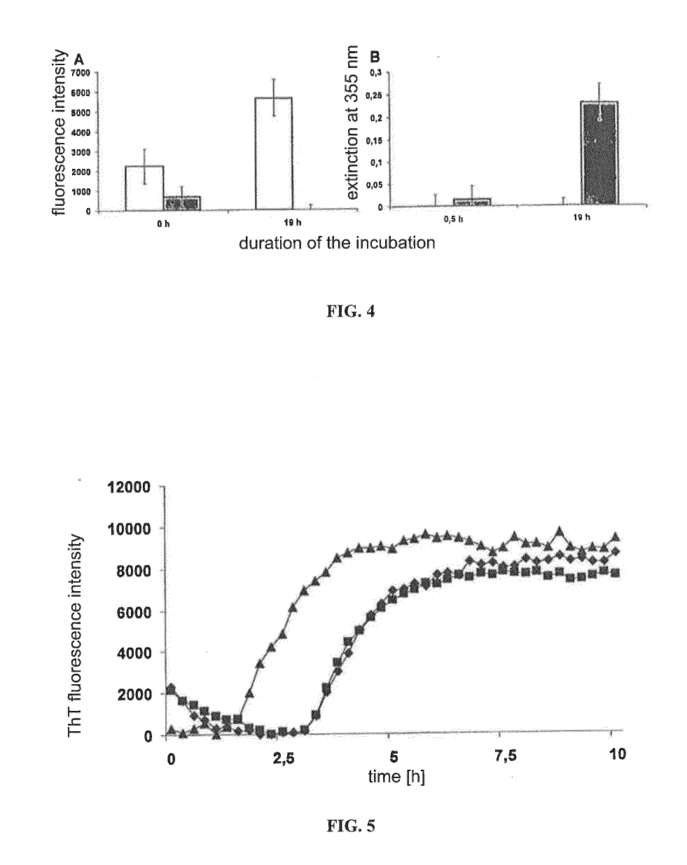

FIG. 5 shows the results of the amyloidogenic properties of Aβ-L3 aggregates, measured by means of ThT fluorescence intensity. Amyloidogenic seeds are particles which act as “nuclei” and expedite the aggregation process. With regard to the aggregation of Aβ, it is known that existing Aβ oligomers/seeds considerably expedite the aggregation process of monomers. For the experiment, seeds were produced which consisted of Aβ (triangles) and such, which consisted of Aβ and L3 (squares). After incubating Aβ and A-L3 mixtures for 5 days, the seeds were centrifuged off and washed. The seeds (20% v/v) were added to freshly prepared Aβ in the ThT test. Aβ without seeds (rhombi) were measured for control purposes. The graph shows the ThT fluorescence over time. Compared to the Aβ solutions without seeds, the seeds containing L3 do not result in any accelerated aggregation. This is an indication that Aβ-D3 aggregates no longer have amyloid structures. Contrary to Aβ seeds, seeds composed of Aβ and L3 do not expedite the Aβ aggregation process.

1. A protein, comprising sequence no. 1.

2. A protein
comprising a sequence section according to sequence no. 1 and by comprising a sequence section that causes secretability of the protein.

3. The protein according to claim 2, comprising sequence no. 2.

4. Deoxyribonucleic acid, coding for a protein according to sequence no. 1.

5. The deoxyribonucleic acid according to claim 4, according to sequence no. 3.

6. Deoxyribonucleic acid, coding for a peptide according to claim 3.

7. The deoxyribonucleic acid according to claim 6, according to sequence no. 4.

8. A vector, containing a sequence section according to sequence no. 3.

9. The vector according to claim 8, comprising sequence no. 5.

10. A vector, containing a sequence section according to sequence no. 4.

11. The vector according to claim 10, comprising sequence no. 6.

12. A pharmaceutical, comprising a protein according to sequence no. 1.

13. A pharmaceutical, comprising a protein which sequence no. 1 and a sequence section which functionally causes secretability.

14. A pharmaceutical, comprising a deoxyribonucleic acid according to any one of claims 4 to 7.

15. A pharmaceutical, comprising a vector according to any one of claims 8 to 11.

16. A method for treating Alzheimer's disease, wherein at least one agent according to claim 1 is introduced into the body.


SEQUENCE LISTING (6 sequences)

SEQ ID NO: 1 - protein, 12 aa, Artificial Sequence
  Description of Artificial Sequence Synthetic peptide
LRMMLQIKRIPR

SEQ ID NO: 2 - protein, 28 aa, Artificial Sequence
  Description of Artificial Sequence Synthetic peptide
ASALLILALVGAAVAVLRMMLQIKRIPR

SEQ ID NO: 3 - DNA, 36 bp, Artificial Sequence
  Description of Artificial Sequence Synthetic oligonucleotide
ctgaggatgatgctgcagatcaagaggatccccagg

SEQ ID NO: 4 - DNA, 84 bp, Artificial Sequence
  Description of Artificial Sequence Synthetic oligonucleotide
gccagcgccctgctgatcctggccctggtgggcgccgccgtggccgtgctgaggatgatgctgcagatcaagaggatccccagg

SEQ ID NO: 5 - DNA, 4713 bp, Artificial Sequence
  Description of Artificial Sequence Synthetic polynucleotide
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattcatggtgctgaggatgatgctgcagatcaagaggatccccagggcccgggatccaccggtcgccaccatggacaacaccgaggacgtcatcaaggagttcatgcagttcaaggtgcgcatggagggctccgtgaacggccactacttcgagatcgagggcgagggcgagggcaagccctacgagggcacccagaccgccaagctgcaggtgaccaagggcggccccctgcccttcgcctgggacatcctgtccccccagttccagtacggctccaaggcctacgtgaagcaccccgccgacatccccgactacatgaagctgtccttccccgagggcttcacctgggagcgctccatgaacttcgaggacggcggcgtggtggaggtgcagcaggactcctccctgcaggacggcaccttcatctacaaggtgaagttcaagggcgtgaacttccccgccgacggccccgtaatgcagaagaagactgccggctgggagccctccaccgagaagctgtacccccaggacggcgtgctgaagggcgagatctcccacgccctgaagctgaaggacggcggccactacacctgcgacttcaagaccgtgtacaaggccaagaagcccgtgcagctgcccggcaaccactacgtggactccaagctggacatcaccaaccacaacgaggactacaccgtggtggagcagtacgagcacgccgaggcccgccactccggctcccagtagagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 6 - DNA, 4758 bp, Artificial Sequence
  Description of Artificial Sequence Synthetic polynucleotide
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattcatggccagcgccctgctgatcctggccctggtgggcgccgccgtggccgtgctgaggatgatgctgcagatcaagaggatccccagggcccgggatccaccggtcgccaccatggacaacaccgaggacgtcatcaaggagttcatgcagttcaaggtgcgcatggagggctccgtgaacggccactacttcgagatcgagggcgagggcgagggcaagccctacgagggcacccagaccgccaagctgcaggtgaccaagggcggccccctgcccttcgcctgggacatcctgtccccccagttccagtacggctccaaggcctacgtgaagcaccccgccgacatccccgactacatgaagctgtccttccccgagggcttcacctgggagcgctccatgaacttcgaggacggcggcgtggtggaggtgcagcaggactcctccctgcaggacggcaccttcatctacaaggtgaagttcaagggcgtgaacttccccgccgacggccccgtaatgcagaagaagactgccggctgggagccctccaccgagaagctgtacccccaggacggcgtgctgaagggcgagatctcccacgccctgaagctgaaggacggcggccactacacctgcgacttcaagaccgtgtacaaggccaagaagcccgtgcagctgcccggcaaccactacgtggactccaagctggacatcaccaaccacaacgaggactacaccgtggtggagcagtacgagcacgccgaggcccgccactccggctcccagtagagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat